(19) United States
(12) Patent Application Publication
(10) Pub. No.: US 2025/0181880 A1
(43) Pub. Date: Jun. 5, 2025

(54) AN EMERGENCY MEDICAL INFORMATION DEVICE

(71) Applicants: Adriana Brusi, Montrose, Victoria (AU); Hendrik Gutwirth, Montrose, Victoria (AU)

(72) Inventors: Adriana Brusi, Montrose, Victoria (AU); Hendrik Gutwirth, Montrose, Victoria (AU)

(21) Appl. No.: 18/846,107

(22) PCT Filed: Mar. 10, 2023
(86) PCT No.: PCT/AU2023/050171

§ 371 Date: Sep. 11, 2024

(30) Foreign Application Priority Data
Mar. 11, 2022 (AU) 2022900596

(51) Int. Cl. G06K 19/077; G06K 19/07; G16H 10/65

(52) U.S. Cl. CPC G06K 19/07758; G06K 19/0723; G16H 10/65

(57) ABSTRACT

An emergency medical information apparatus for providing information relating to a person who is an occupant of a vehicle, the device comprising an apparatus body adapted to releasably attach to a seat belt of a vehicle in proximity to the person as the occupant; a wireless communicator mounted in the apparatus body and having a storage element for providing information relating to the occupant of the vehicle; and a shortrange transmitter able to communicate the information about the occupant to a nearby receiver without any instigation by the occupant wherein a person providing assistance can readily obtain at least some information from the wireless communicator which is relevant to the person as the occupant enabling providing of appropriate informed assistance.

### CROSS-REFERENCE TO RELATED APPLICATIONS

This application is a 371 National Phase Entry of International Patent Application No. PCT/AU2023/050171 filed on Mar. 10, 2023, which claims the benefit of Australian Provisional Patent Application No. 2022900596 filed on Mar. 11, 2022, the contents of which are incorporated herein by reference in their entirety.

### FIELD OF THE INVENTION

The present invention relates to an emergency medical information device for use regarding an occupant of a vehicle.

The invention has been developed primarily for use with seat belts and particularly seat belts on child seats or baby capsules or disabled or special needs persons for providing medical information to first responders and will be described hereinafter with reference to this application. However, it will be appreciated that the invention is not limited to this particular field of use.

### BACKGROUND OF THE INVENTION

Personal emergency information can be provided in display items such as medical identification bracelets or necklaces. These usually relate to a single medical issue and are worn to inform medical personnel of specific health conditions such as allergies, epilepsy, asthma, and Alzheimer's disease. These are of prime consideration to first responders when the wearer is nonresponsive or unable to communicate.

However, such items need to be located on the person. They can readily be lost or no longer on a person in a car accident. They could be destroyed by water or fire. They could merely be damaged but therefore still be rendered ineffective and of no use to the first responder.

Some medical information could be on a card that is in a person's wallet or purse. However, unless the person provides such a card a first responder is not going to know to look as it is not a common or usual case. Similarly, a person could have information on a mobile phone, but this is not available to others due to security access on phones and not having information in a known position.

A particular form of information source can be a piece of jewelry such as a necklace or bracelet. These are used by particular medical organizations to entice users to wear them for aesthetic purposes and to encourage usage so there are more people providing ready information. Apart from the problem of the users forgetting to wear them, often such devices are hazardous to the wearer. They could be a choke hazard, or strangling hazard. This is particularly the case for a person with limited control due to physical or mental complications. It is definitely the case for children and infants and some disabled persons who do not have an inbuilt sense of hazard awareness.

Therefore, many cases exist where such approaches are not appropriate, not effective, and not used.

The effect of a first responder at an accident or incident having a lack of information about a person they are trying to assist can result in an inappropriate assistance, an incorrect medical procedure or a significant loss of time trying to determine the underlying condition of the person before undertaking an appropriate action. These delays, mistakes, or inappropriate assistance can be through the range deleterious to the person from being awkward or embarrassing to being critical or fatal.

Seat belts are important assets in providing a safety benefit in a vehicle and particularly in the event of an accident. It is therefore critical to not affect the working of the seat belt.

It can be seen that known prior art devices for providing information relating to the occupant of a vehicle has the problems of:
(a) limited amount of space on which information can be provided;
(b) requiring active participation of the person needing attention to provide critical information;
(c) devices being hazardous;
(d) devices being wearable and readily forgotten;
(e) risk that the information provided will be destroyed in an emergency; and
(f) no validation that the available information relates to a particular occupant of the vehicle.

The present invention seeks to provide an apparatus device for providing information relating to the occupant of a vehicle, which will overcome or substantially ameliorate at least one or more of the deficiencies of the prior art, or to at least provide an alternative.

It is to be understood that, if any prior art information is referred to herein, such reference does not constitute an admission that the information forms part of the common general knowledge in the art in Australia or any other country.

### SUMMARY OF THE INVENTION

According to an embodiment of the present invention, there is provided a device for providing information relating to the occupant of a vehicle, the device comprising: a body adapted to releasably attach to part of a vehicle in proximity to the occupant; a wireless communicator comprising storage for storing information relating to an occupant of the vehicle; and a transmitter for transmitting the stored information; and a visual indicator on the body to indicate the location of the wireless communication device.

In one form the invention provides an emergency medical information apparatus for providing information relating to a person who is an occupant of a vehicle, the device comprising an apparatus body adapted to releasably attach to a seat belt of a vehicle in proximity to the person as the occupant; a wireless communicator mounted in the apparatus body; the wireless communicator including a storage element for storing information relating to the occupant of the vehicle; and a shortrange transmitter able to communicate the stored information about the occupant to a nearby receiver without any instigation by the occupant. This allows a person providing assistance to readily obtain at least some information from the wireless communicator which is relevant to the person as the occupant enabling providing of appropriate informed assistance.

The body may be a seat belt cover adapted to releasably attach to a seat belt.

The wireless communicator may be a near-field communications device.

The information stored on the device may be an identifier linking to additional data stored remotely. The information stored may be medical information.

It can be seen that the invention of a device for providing information relating to the occupant of a vehicle provides the benefit that first responders to the scene of an accident can retrieve important medical information relating to the occupant when the occupant is nonresponsive or unable to communicate.

According to another aspect of the invention there is provided a system for providing information relating to the occupant of a vehicle, the system comprising:
the device for providing information relating to the occupant of a vehicle as described above; a wireless reader configured to read the information stored on the wireless communicator; and a display for displaying the information relating to the occupant of the vehicle.

The system may comprise remote digital storage for storing information related to the occupant of the vehicle.

The system may further comprise a secondary visual indicator for informing first responders of the presence of the device for providing information.

The wireless reader and/or the display may take the form of a mobile device.

Also, the invention provides a method of aiding first responders provide suitable aid to a user including the steps of:
creating localized open access data about a user;
loading data on an NFC in a secure, protected device with NFC location indicator;
removably mounting the secure protected device to a seat belt used by the relevant person;
allowing first responders and others to locate and locally communicate with NFC device by NFC reader; and
allowing first responders to assist with aid of the appropriate open access information.

It can be seen that the invention of a system for providing information relating to the occupant of a vehicle provides the benefits of at least one or a combination of more than one of:
(a) giving first responders immediate open localized access to crucial medical information when the occupant is nonresponsive or unable to communicate;
(b) providing a safe hazard free storage of communication device so no sharp edges, no access to avoid any possibility of choking hazard, and no deformation or damage to device;
(c) providing a means where the information is unlikely to be destroyed or displaced or damaged;
(d) providing information localized to an occupant at a particular location in a vehicle;
(e) not affecting normal safety operations of vehicle safety devices including not affecting seat belt operation; and
(f) providing options of including broadcast imaging on device or not.

Other aspects of the invention are also disclosed.

### BRIEF DESCRIPTION OF THE FIGURES

Notwithstanding any other forms which may fall within the scope of the present invention, preferred embodiments of the invention will now be described, by way of nonlimiting example only, with reference to the accompanying drawings in which:

FIG. 1 is a perspective view of a seat belt cover forming an emergency medical information apparatus for providing information relating to a person who is an occupant of a vehicle in accordance with a first embodiment of the present invention;

FIG. 2 is a front diagrammatic view of the seat belt cover that can form the first embodiment of FIG. 1;

FIG. 3 is a front photographic view of a short distance wireless communicator in the form of a near-field communication (NFC) device that can be used in the emergency medical information apparatus of FIG. 1 or other embodiments;

FIG. 4 is a perspective view of a seat belt cover forming an emergency medical information apparatus for providing information relating to a person who is an occupant of a vehicle in accordance with a second embodiment of the present invention;

FIGS. 5 and 6 are end view and rear view of the emergency medical information apparatus of FIG. 4;

FIGS. 7 and 8 are diagrammatic representations of a seat belt cover of FIG. 1 or FIG. 4, in accordance with some embodiments, when attached to a seat belt and for providing information in relation to the occupant of the vehicle such as the driver or a child on a riser seat in accordance with some embodiments of the invention;

FIGS. 9, 10, and 11 are diagrammatic exploded view and cutaway view and rear view of seat belt cover of FIG. 4 showing the mount and locatability of the NFC in a hazard preventing manner;

FIG. 12 is a diagrammatic view of the relevant size and location of the NFC with regard the seat belt so as to avoid any hindrance with seat belt usage avoidance of edge hazards and allowing ready short distance wireless communication to a mobile device;

FIGS. 13 and 14 are diagrammatic views of the operative features of two embodiments of an NFC device for use in the emergency medical information apparatus with each having an open access format allowing always for open access and in a second embodiment a further category being a secure access; and

FIG. 15 is a diagrammatic block diagram of a method of aiding first responders provide suitable aid to a user in accordance with one or more embodiments of the invention.

### DETAILED DESCRIPTION OF THE INVENTION

It should be noted in the following description that like or the same reference numerals in different embodiments denote the same or similar features.

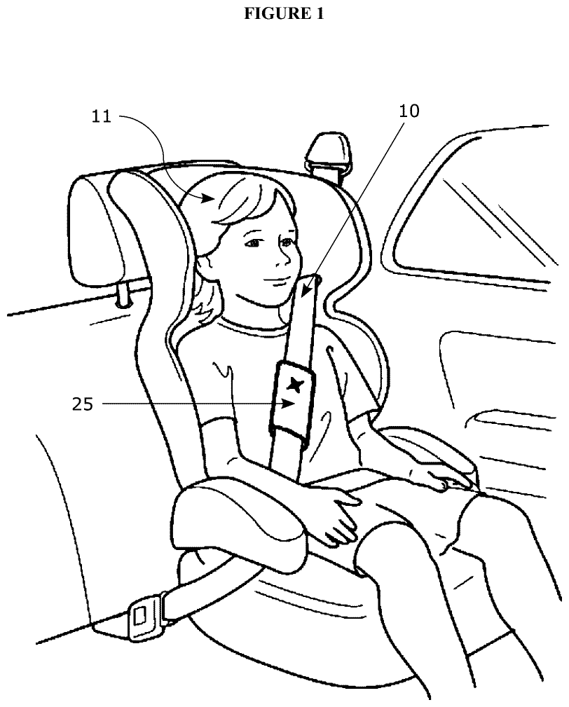

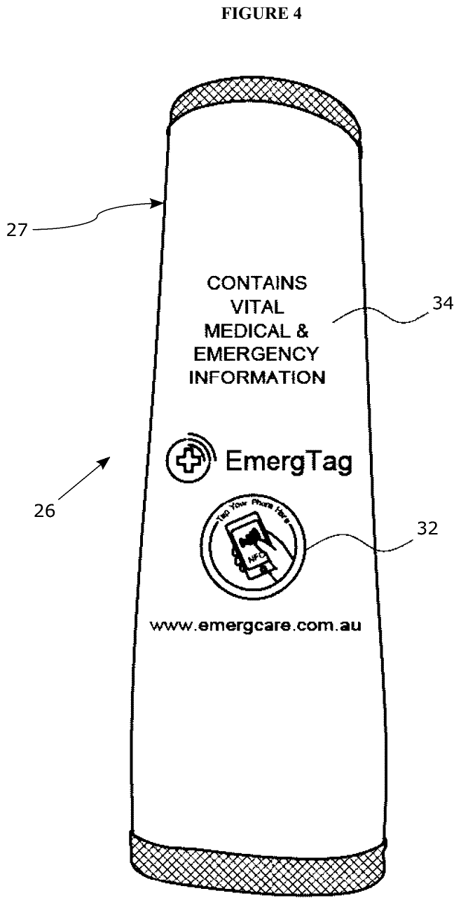

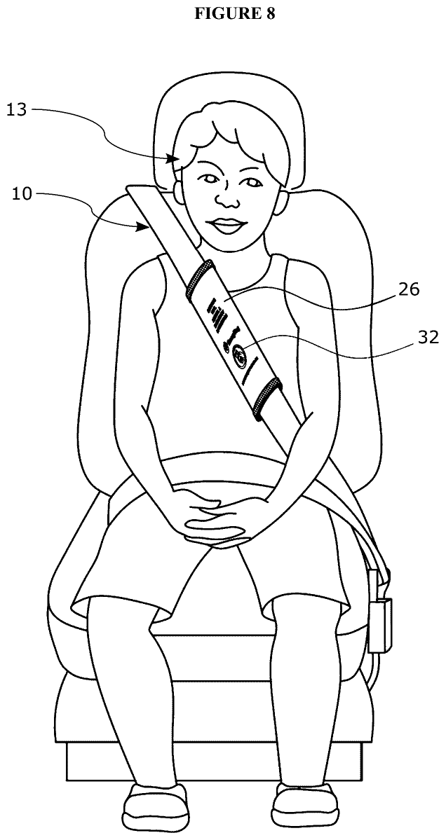

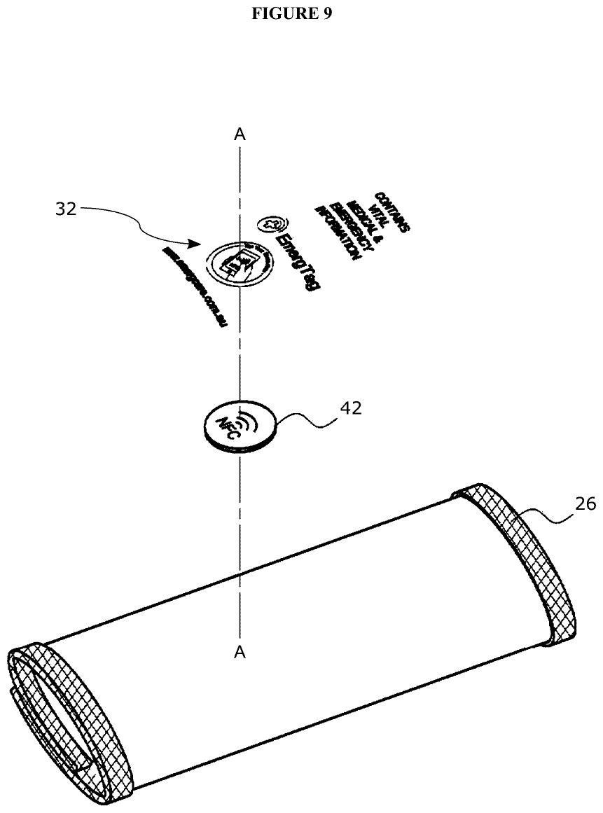

Referring to the drawings, there are shown two embodiments of an emergency medical information device. There is a first embodiment of the emergency medical information device (25) of FIGS. 1, 2, and 3 which includes a broadcast of personal information addition. There is a usual second embodiment of the emergency medical information device (26) of FIGS. 4, 7, and 8 which does not include a broadcast of personal information addition.

In each embodiment, the structure of the emergency medical information device (25, 26) is in the form of a seat belt cover (27) for providing information relating to the occupant of a vehicle at that location. The seat belt cover (27) comprises a body of protective material (28) and possibly edging (29) with the cover adapted to releasably attach to the seat belt (10) of the occupant. The seat belt cover (27) forming the first or second form of emergency medical information devices (25, 26) housing a wireless communicator (42) that and open access digital storage for storing information relating to the occupant and a short field transmitter for transmitting over short distance to the stored information. For the embodiments depicted, the wireless communicator is a near-field communications (NFC) device that transmits only over a short distance under about 20 cm. However, it is readily transmittable through most permeable materials. Fundamentally the best reception is achieved by the NFC reader such as a mobile phone (51) as shown in FIG. 12 being placed directly over the NFC.

In the first emergency medical information device (25) of FIGS. 1, 2, and 3 there is a version for a special needs child (11). This version operates similarly to the second embodiment and its operation will be described later. However, this version takes the approach of adding broadcast of personal information.

Clearly with assisting special needs children there is usually a required modified social communication element and a treatment element. It can be beneficial to have a broadcast of personal information so there is immediate understanding of the possible variations of personal communication that will be required while attending to treatment element.

In this regard the first emergency medical information device (25) of FIGS. 1, 2, and 3 can include broadcast of personal information such as a special needs indicator (31) that directly advises people coming to the aid. It can be beneficial in some instances but in others the special needs person might find it upsetting. Another broadcast of personal information can be special needs information (33).

In the second version of the invention there is not the use of the broadcast of personal information. This system is for all users including special needs children and relies on a passive provision of information without the broadcast of information by external broadcast of personal information. As shown in FIGS. 1, 7, and 8 the emergency medical information device (26) can be for a special needs person, a child or adult, a driver, and/or a passenger.

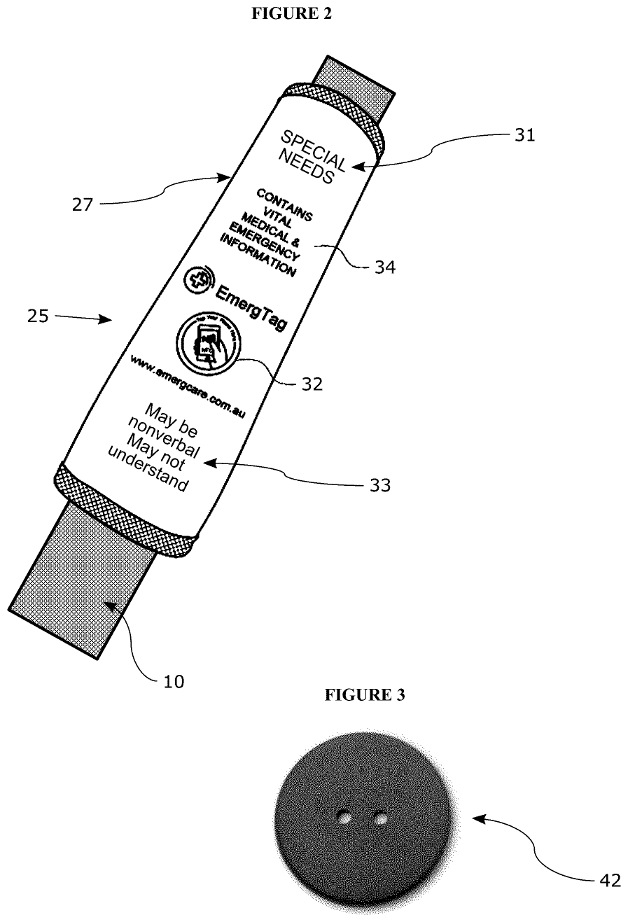

Referring to FIG. 2 or 4 the seat belt cover (27) forming the first or second form of emergency medical information device (25, 26) can include various indicators including:
(a) a special needs indicator (31) identifying that the occupant has special needs;
(b) special needs information (33) such as the person is not verbally communicable; and/or
(c) general advice indicator (34) advising the information that the device includes vital medical and emergency information.

Referring to figures, the seat belt cover (27) forming the first or second form of emergency medical information device (25, 26) houses the wireless communicator (42) which is provided with a visual indicator (32) forming an NFC locator on the outside body to indicate the location of the wireless communication device in the form of a circular NFC device (42).

Referring to FIGS. 3 and 12, there is shown how the NFC device itself is chosen to be circular with rounded edges so as to avoid having any sharp edges. Further as shown in FIG. 12 the NFC device (42) is sized and located to be smaller than the width of the seat belt (10) and to lie flat thereon so that no edges are felt by the user when strapped into the seat belt (10) in use.

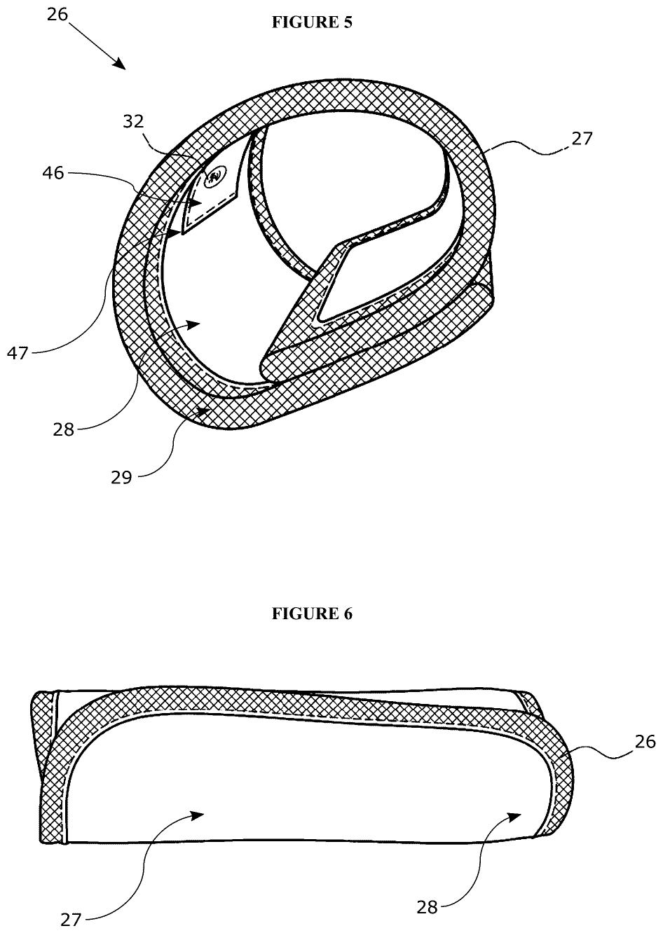

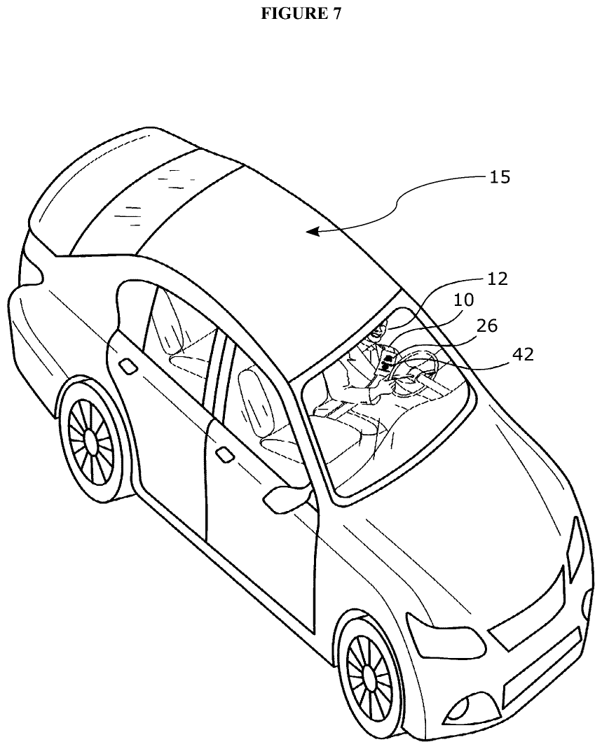

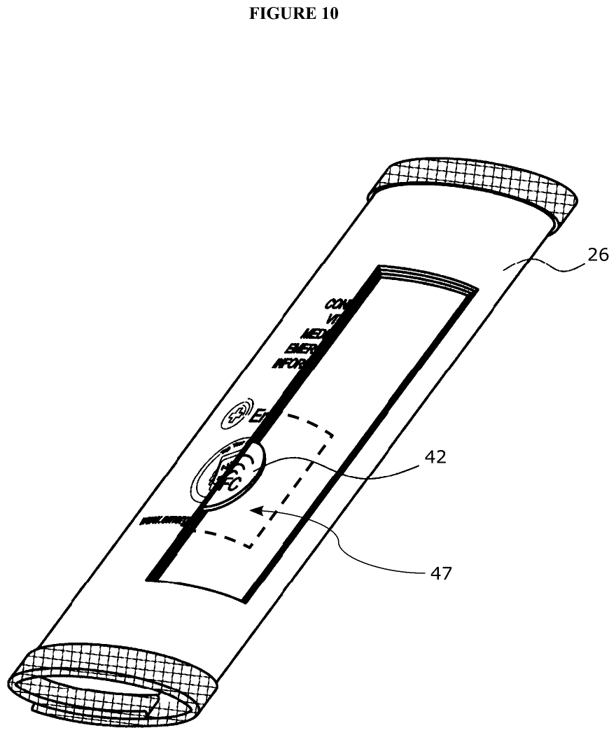

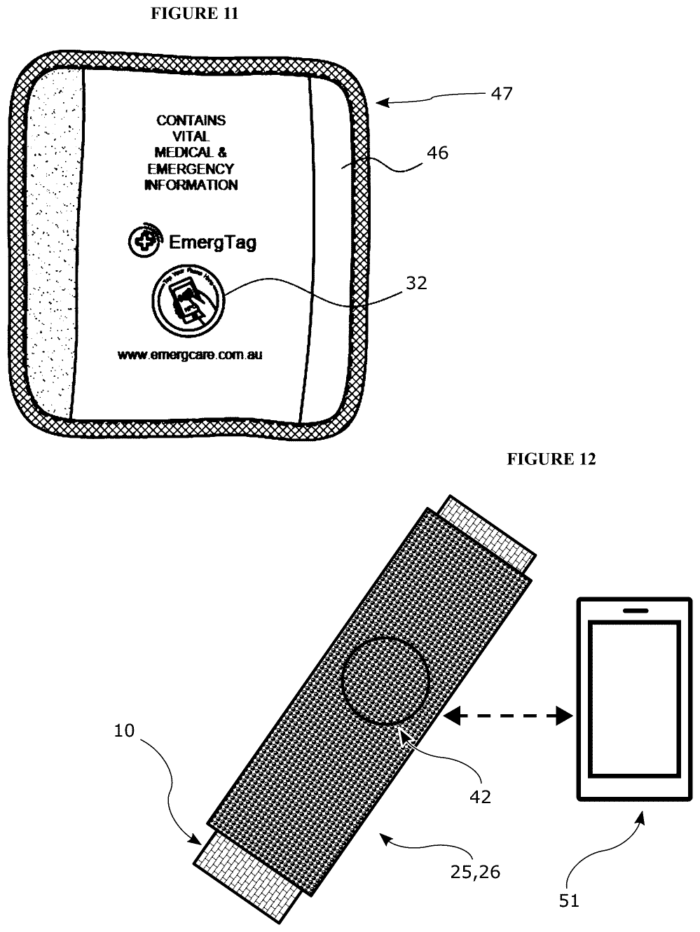

Referring to FIGS. 5, 10, 11, and 12 there is shown how the NFC device is enclosed within the seat belt cover (27) to be at a position and without access so as to eliminate access hazards. This is achieved as shown in FIGS. 5 and 10 by being wrapped in the protective material (28) of the seat belt cover (27) such as neoprene. There can be triple stitching (47) of the protective material around the NFC to further prevent any movement from the required alignment position on the seat belt (10) and to prevent any access. A patch (46) could also be triple stitched in position from an inside surface or within a layer of the wrap of protective material (28). The protective material (28) and protective patch could be other strong flexible material that is impenetrable and suitable such as Kevlar sheeting.

In the manufacturing of the accessory a small incision could be made behind the NFC locator (32) with a pocket that holds the NFC that is sealed. A second layer of neoprene (46) overlays and is sealed around with the entire accessory hemmed around all the edges (47). The NFC is held in place underneath because it is a firm pocket that holds the NFC against the logo.

The NFC tag is not accessible as it sits behind multi layers of material that would require a knife of scissors to physically remove it. Making it tamper proof and safe for children.

However, the device is effective when a mobile device (51) is held over the NFC locator (32) and thereby the NFC (42) activates.

There are no zips, threads, ability, for the NFC to come loose. Even if the sides of the accessory in some manufacturing mishap was to come apart, the NFC chip is still secured in another layer, protecting anyone from any potential hazard.

The information stored can be an identifier linking to additional data stored remotely. Alternatively or in combination, the information stored can be detailed information about the occupant, for example medical information. Further examples of the information that may be stored are:
(a) an image of the occupant for verifying the information relates to the actual occupant;
(b) name of the occupant;
(c) next of kin/contact number;
(d) medical conditions, allergies, or any other information relevant to first responders;
(e) an identification code which may be used for linking to additional external data;
(f) full list of medications;
(g) any contraindication (no penicillin, religious exclusions);
(h) blood type;
(i) organ donor status; and
(j) do not resuscitate status.

The emergency medical information apparatus includes an open access category of information wherein the person providing assistance can access all of the information in that open access category.

As shown in FIG. 13 the NFC (42) includes a communicator (coms) for undertaking the near-field communication and connecting to the reader and writer on the NFC such that the information is written to the NFC and able to be read from the NFC by way of the controller (contr).

This information is written and read from an open access storage. Therefore, in use the wireless communicator provides an unimpeded communication access wherein access or transmittal of the information included in the one access category of information is readily available to any person providing assistance, such as a first responder.

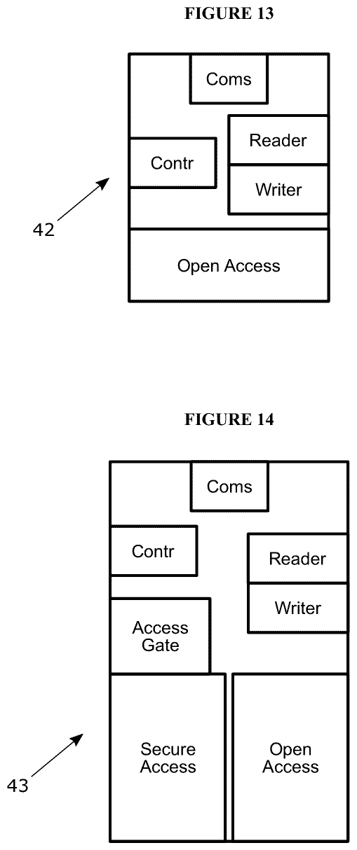

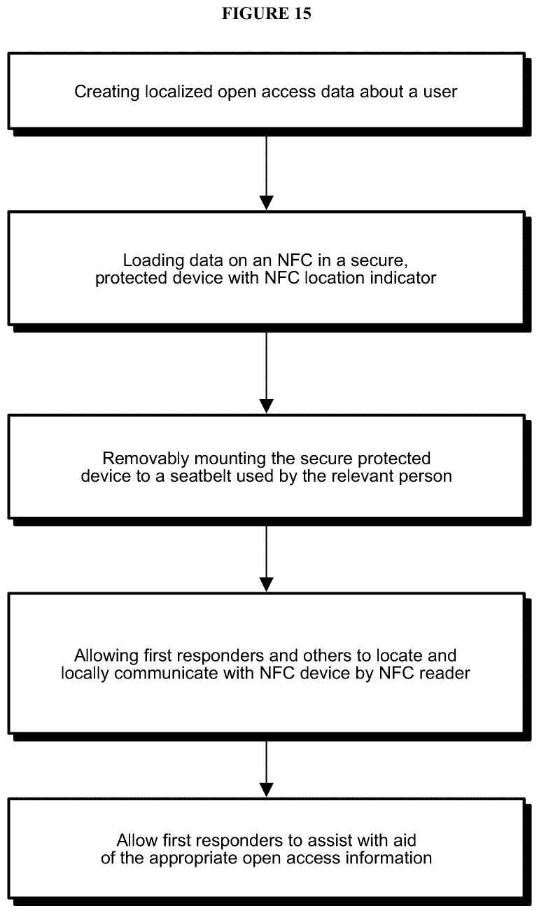

As shown in FIG. 14 the NFC (43) includes a communicator (coms) for undertaking the near-field communication and connecting to the reader and writer on the NFC such that the information is written to the NFC and able to be read from the NFC by way of the controller (contr). There is clear open access through the controller. However, there is also an access gate to a second secure access storage. The controller can control the opening of the access gate so that there is access to the secure access.

In use, therefore with the form of the emergency medical information apparatus (43), the person providing assistance can always access the information in the first open access category and can only access others of the plurality of access categories including the information being secure access categories upon authorization.

The wireless communicator and particularly the access gate can act with the controller as a security access wherein access or transmittal of the information included in the other of the plurality of access categories of information is only available to an authorized person having an authorizing code.

An official first responder providing assistance always has access to the open access information and as a person providing assistance can use a contact detail from the open first access category to obtain an authorizing code that enables access or transmittal of the information in one or more of the other access categories of information.

The open access information available to all as against the other information that is secure access information could be separated as follows:

### (A) Open Access Information

An image of the occupant for verifying the
information relates to the actual occupant
Name of the occupant
Next of kin/contact number
Medical conditions, allergies, or any other
information relevant to first responders
Blood type

### (b) Secure Access Information

An identification code which may be used
for linking to additional external data

| Full list of medications | Organ donor status |
|---|---|
| Any contra indication (no penicillin, | Do not resuscitate status |
| religious exclusions) | |

The benefits of such a multiple category access system are that there can be a choice provided to the users of the amount of information available in the open access category that is available in all forms. Further the multiple category access system allows for ready separation and control of information.

While described with reference to attaching to a seat belt (10), the seat belt cover can be versatile and adaptable to fit to other equipment in proximity to an occupant or wearer. For example, the wrap around Velcro adhesive body which forms a sleeve, allows the device to be attached to parts of, for example, baby capsules, prams, bicycles, trikes, plane belts, boats seats, backpacks, and luggage.

Referring to FIG. 4, an example of a system for providing information relating to the occupant of a vehicle is generally indicated by reference numeral (26). The system comprises the seat belt cover (27) as described above to provide information relating to the occupant of a vehicle. The system further comprises a wireless reader configured to read the information stored on the wireless communicator and/or NFC locator (32) and a display for displaying the information relating to the occupant (12, 13) of the vehicle (15).

The system may further comprise remote digital storage for storing information related to the occupant of the vehicle. When communicating with the wireless communicator the wireless reader may receive an identification code linking the operator to remotely stored medical information of the occupant.

The NFC (42, 43) can be used independent of Internet access. Information can be stored both locally. In the event of an emergency no Internet access is required so first responders when accessing the device (26) can receive information stored locally on the device via, for example, a mobile device (51). For example, when in use a first responder to the scene of an emergency can access detailed medical information of the occupant by scanning the wireless device (42, 43) within the seat belt cover with their mobile phone (51).

The importance of the emergency medical information apparatus of the invention is that it is clearly related to emergency related to the particular occupant of the vehicle at that location. It is able to:
(a) have a location that identifies relevance to the occupant of the vehicle at that position;
(b) not affect the normal safety of the occupant by not affecting but enhancing seat belt;
(c) remain in a standard position for the first responders to know where to look and to be in front of mind; and
(d) provide relevant information that is relevant to the occupant at that position when in a vehicle emergency and therefore the entire medical records.

### EXAMPLES

### Example 1

If the occupant of the vehicle is a special needs person that might be limited in communication skills, this information is transmitted initially to the first responder.

Therefore, instead of the first responder assessing that the lack of communication is a trauma effect of the emergency and undertaking a medical treatment which could be incorrect, the first responder is able to receive the information that the occupant is usually limited in responsive communication and be provided with information to manage the communication.

This results in better medical assistance and also in limiting the trauma to the occupant by communicating or treating the occupant in a much more appropriate manner.

### Example 2

If the occupant of vehicle is a person with multiple health issues, the person could have a medical bracelet which is a contact to an external site with access to the complete complex medical history. This information is critical to a professional medical person in a hospital who has access and time and expertise to trawl through all of the information and assess which is the relevant information. However, a first responder does not have that capability.

Therefore, due to the emergency medical information apparatus being able by its shape, position, and communication device to be limited to the vehicle and in particular to emergencies while in the vehicle and more importantly to the occupant of the vehicle at that position and to be an active device to provide information, the first responder is provided the relevant information the first responder first needs to undertake the first emergency actions. It can still have access to the secondary more detailed information for later use.

### Example 3

If the occupant is not fully physically controllable, such as an infant or an epileptic or tantrum-prone or other issue, then it is dangerous to have a chain or bracelet with medical data. Such a device can add to possible injuries.

However, due to the emergency medical information apparatus being able by its shape, position, and communication device to be limited to the vehicle and in particular to emergencies while in the vehicle and more importantly to the occupant of the vehicle at that position and to be an active device to provide information, the first responder is provided the relevant information the first responder first needs to undertake the first emergency actions. It can still have access to the secondary more detailed information for later use.

### Interpretation

### EMBODIMENTS

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures, or characteristics may be combined in any suitable manner, as would be apparent to one of ordinary skill in the art from this disclosure in one or more embodiments.

Similarly, it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Different Instances of Objects

As used herein, unless otherwise specified the use of the ordinal adjectives “first,” “second,” “third,” etc., to describe a common object, merely indicate that different instances of like objects are being referred to and are not intended to imply that the objects so described must be in a given sequence, either temporally, spatially, in ranking, or in any other manner.

### Specific Details

In the description provided herein, numerous specific details are set forth. However, it is understood that embodiments of the invention may be practiced without these specific details. In other instances, well-known methods, structures, and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward,” “rearward,” “radially,” “peripherally,” “upwardly,” “downwardly,” and the like are used as words of convenience to provide reference points and are not to be construed as limiting terms.

### Comprising and Including

In the claims which follow and in the preceding description of the invention, except where the context requires otherwise due to express language or necessary implication, the word “comprise” or variations such as “comprises” or “comprising” are used in an inclusive sense, i.e., to specify the presence of the stated features but not to preclude the presence or addition of further features in various embodiments of the invention.

Any one of the terms “including” or “which includes” or “that includes” as used herein is also an open term that also means including at least the elements/features that follow the term but not excluding others. Thus, including is synonymous with and means comprising.

### SCOPE OF INVENTION

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulae given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the transport accident and medical and emergency first respondent industries.

What is claimed is:

1. An emergency medical information apparatus for providing information relating to a person who is an occupant of a vehicle, the device comprising:
a. an apparatus body adapted to releasably attach to a seat belt of a vehicle in proximity to the person as the occupant;
b. a wireless communicator mounted in the apparatus body;
c. the wireless communicator including:
i. a storage element for providing information relating to the occupant of the vehicle; and
ii. a shortrange transmitter able to communicate the information about the occupant to a nearby receiver without any instigation by the occupant; and
wherein a person providing assistance can readily obtain at least some information from the wireless communicator which is relevant to the person as the occupant enabling providing of appropriate informed assistance.

2. (canceled)

3. (canceled)

4. An emergency medical information apparatus as claimed in claim 1 wherein the apparatus body is a seat belt cover adapted to releasably attach to a seat belt securing the person as an occupant in the vehicle.

5. (canceled)

6. An emergency medical information apparatus as claimed in claim 4 wherein the closing means is formed of soft material to not provide any effect on the operation of the seat belt and not form a hazard to the person as an occupant.

7. An emergency medical information apparatus as claimed in claim 6 wherein the closing means includes at least one hook-and-loop closure element.

8. An emergency medical information apparatus as claimed in claim 6 wherein the closing means includes a frangible element allowing fixed location of the seat belt wrap on the seat belt to retain in position over an extended period relating to multiple trips for a person that is the only person to occupy that location and seat belt in the vehicle and wherein removal can occur upon breaking the frangible element.

9. (canceled)

10. (canceled)

11. (canceled)

12. (canceled)

13. An emergency medical information apparatus as claimed in claim 1 wherein the wireless communicator is a near-field communications (NFC) device.

14. An emergency medical information apparatus as claimed in claim 13 wherein the NFC device is substantially planar, has rounded edges, and is substantially circular.

15. (canceled)

16. (canceled)

17. An emergency medical information apparatus as claimed in claim 13 wherein the NFC device is mountable parallel to the planar width of the seat belt and is sized less than the lateral width of a seat belt allowing the NFC device to overlay the seat belt in a flat manner when in location.

18. (canceled)

19. An emergency medical information apparatus as claimed in claim 13 wherein the NFC device is sealed within the apparatus body preventing physical access and thereby eliminating the device as a hazard to the person as the occupant.

20. An emergency medical information apparatus as claimed in claim 13 wherein the NFC device is substantially surrounded by cushioning material to further eliminate the device as a hazard to the person as the occupant.

21. An emergency medical information apparatus as claimed in claim 13 wherein the NFC device is overlayed by an indicating sign on an exterior outwardly facing surface of the apparatus body to provide indication for allowing optimizing NFC connection by positioning of a receiving NFC communicating device.

22. An emergency medical information apparatus of claim 1 wherein the information stored is available locally from the wireless communicator mounted in the apparatus body without requirement for external remote connection.

23. An emergency medical information apparatus of claim 22 wherein the information stored is modifiable by use of a mobile device linking to the wireless communicator in a localized manner.

24. An emergency medical information apparatus of claim 22 wherein the information stored is modifiable at one or more times by a remote connection to an online site and linking to the wireless communicator in a localized manner enabling the modified information to be available at all times locally from the wireless communicator without requirement for external remote connection.

25. (canceled)

26. An emergency medical information apparatus according to claim 22 wherein the information can include one or more of:
a. your name;
b. allergy information;
c. your next of kin's name/contact number;
d. next of kin's address;
e. next of kin's e-mail address;
f. any medical condition you have;
g. full lists of medications;
h. any contraindications (no penicillin, etc.);
i. blood group;
j. organ donation; and
k. do not resuscitate (DNR) status.

27. (canceled)

28. (canceled)

29. (canceled)

30. (canceled)

31. An emergency medical information apparatus according to claim 1 wherein the wireless communicator includes a security access wherein access or transmittal of the information included in the other of the plurality of access categories of information is only available to an authorized person having an authorizing code such as an official first responder providing assistance always having an authorizing code that enables access or transmittal.

32. (canceled)

33. An emergency medical information apparatus according to claim 31 wherein person providing assistance can use a contact detail from the open first access category to obtain an authorizing code that enables access or transmittal of the information in one or more of the other access categories of information.

34. A system for providing information relating to an occupant of a vehicle, the system comprising:
a. an emergency medical information apparatus for providing information relating to the occupant of a vehicle, the emergency medical information apparatus comprising an apparatus body adapted to releasably attach to a seat belt of a vehicle in proximity to the person as the occupant and a wireless communicator mounted in the apparatus body, the wireless communicator including a storage element for providing information relating to the occupant of the vehicle and a shortrange transmitter able to communicate the information about the occupant to a nearby receiver without any instigation by the occupant;
b. a handheld communicating device complementary to the wireless communicator of the emergency medical information apparatus configured to receive the information stored on the wireless communicator; and
c. a display for displaying the information relating to the occupant of the vehicle wherein the handheld communicating device is a wireless reader or wherein handheld communicating device complementary to the wireless communicator of the emergency medical information apparatus operate on NFC communication.

35. (canceled)

36. (canceled)

37. (canceled)

38. The system as claimed in claim 34 wherein the wireless reader and/or the display takes the form of a mobile device.

39. (canceled)

40. An emergency medical information apparatus for providing information relating to a person who is an occupant of a vehicle, the device comprising:
a. an apparatus body adapted to releasably attach to a seat belt of a vehicle in proximity to the person as the occupant;
b. a wireless communicator mounted in the apparatus body;
c. the wireless communicator including:
i. a storage element for providing information relating to the occupant of the vehicle; and
ii. a shortrange transmitter able to communicate the information about the occupant to a nearby receiver without any instigation by the occupant;
wherein
a. the provided information is created localized open access data about a person who is an occupant of a vehicle which is loading data on an NFC as the shortrange transmitter and removably mounted the secure protected device to a seat belt used by the relevant occupant of the vehicle;
b. allowing the person such as the first responders and/or others to locate and locally communicate with NFC device by an NFC reader;
c. wherein the person such as the first responder can access appropriate informed assistance with the aid of the appropriate open access information which is relevant to the person as the occupant; and
wherein the person such as the first responder providing assistance can readily obtain at least some information from the wireless communicator, which is relevant to the person as the occupant, enabling providing of appropriate informed assistance.
